(19) United States
(12) Patent Application Publication
(10) Pub. No.: US 2023/0088767 A1
(43) Pub. Date: Mar. 23, 2023

(54) CENTRIFUGAL MICROFLUIDIC DEVICE WITH BLOCKING AND DETECTION CHAMBERS

(71) Applicant: BLUESENSE DIAGNOSTICS APS, Copenhagen (DK)

(72) Inventors: Jeppe FOCK, Copenhagen (DK); Shuo ZHANG, Copenhagen (DK); Tony LIAO, Copenhagen (DK); Melanie BADE, Copenhagen (DK); Marco DONOLATO, Copenhagen (DK); Lars NIELSEN, Copenhagen (DK); Joan VIEYRA, Copenhagen (DK); David HERSI SMITH, Copenhagen (DK); Michael CREAGH, Copenhagen (DK); John MCCORMACK, Copenhagen (DK)

(73) Assignee: BLUESENSE DIAGNOSTICS APS, Copenhagen (DK)

(21) Appl. No.: 17/792,052

(22) PCT Filed: Jan. 19, 2021
(86) PCT No.: PCT/EP2021/050990

§ 371 Date: Jul. 11, 2022

(30) Foreign Application Priority Data
Jan. 20, 2020 (SE) 2050042-7
Jan. 20, 2020 (SE) 2050043-5
Jun. 12, 2020 (SE) 2050707-5

(51) Int. Cl. G01N 33/569; G01N 21/82; G01N 21/07; B01L 3/00

(52) U.S. Cl. CPC G01N 33/56983; G01N 21/82; G01N 21/07; B01L 3/502761; G01N 2021/0328

(57) ABSTRACT

A fluidic device for detecting a target molecule in a fluid sample comprising a blocking chamber in fluidic communication with a detection chamber forming a blocking-detection chamber pair, the blocking chamber provided with at least one reagent for binding a non-target molecule in the sample, the blocking chamber adapted to maintain at least a portion of the bound non-target molecule within the blocking chamber, the detection chamber comprising at least one reagent for binding a target-molecule such that the target-molecule may be detected. The device comprises a combination detection chamber adapted to receive at least one reagent for binding both target and non-target molecules such that the combination of target and non-target molecules may be detected by binding to a detector.

### FIELD OF THE INVENTION

The present disclosure relates to a centrifugal microfluidic device. In particular, it relates to a centrifugal microfluidic device comprising a blocking and detection chamber and a combination detection chamber, and a method for detecting a target molecule in a fluid sample.

### BACKGROUND OF THE INVENTION

Microfluidic devices are for the processing and handling liquids in the nanolitre to microlitre range. Microfluidic devices comprise fluidic chambers for receiving and processing, retaining, and/or holding liquids, and channels for the delivery and routing of liquids throughout the device. Centrifugal microfluidic devices are a type of microfluidic device whereby centrifugal forces are used to pump fluids through the device.

Centrifugal microfluidic devices are rotated such that a centrifugal force acts upon the liquid in the device. Typical for centrifugal microfluidic devices are passive valves which use the surface tension of the liquid within the channels and chambers to create capillary stops, sometimes described as fluidic seals or closures, generally at regions whereby the channel geometry is altered, such as rapidly widened. The device is rotated to generate a centrifugal force sufficient to break such capillary stops and in such a manner the flow of fluid through the device can be sequenced and time-controlled.

The flow of fluid through the microfluidic device when the device is not rotating may be achieved in different manners. Siphons that prime based on capillary action are one such manner. However, such siphons may not be sufficiently reliable, or efficient on their own. This is especially so when the surface tension of the fluids may vary, impacting the rate of capillary wicking in the siphons. Improved devices and techniques for the flow of fluids in passive siphons would be advantageous.

Centrifugal microfluidics is especially used in the life sciences, in particular in lab based analytics and diagnostics. As operations such as pipetting, mixing, measuring, aliquoting and centrifuging are possible to automate via centrifugal microfluidics it is especially relevant in environments where such operations may be difficult to control such as small-scale or remote laboratories lacking traditional and expensive analytic devices.

In once such analytic/diagnostic process centrifugal microfluidic devices may be used for the detection of target molecules in a sample. A common problem in the detection of target molecules is non-specific binding of a detector molecule to non-target molecules. Blocking of non-target molecules is one solution to the problem. However, the concentration of blocking agent with respect to the concentration of non-target molecules needs to controlled precisely. The amount of blocker in relation to the substance to be blocked has to be precisely controlled to avoid any saturation effect. The provision of too much blocking agent with respect to the concentration of molecules to be blocked may lead to soluble complexes of blocking agent bound to itself and not sufficiently to the molecule to be blocked. Too little blocking agent would clearly lead to insufficient blocking and the presence of a greater amount of non-target molecule capable of interfering with the detection process. This is related to the precipitation curve, a concept known in immunology where the ratio of antigen to antibody can be divided in to three zones: a prozone where there is an abundance of antigen, an equivalence zone where there is maximum precipitation, and a postzone where there is an abundance of antibody. This concept is traditionally described in terms of antigen-antibody concentrations, however, it is equally relevant in antibody-antibody immunoprecipitation. As the concentration of the non-target molecule may not be known in advance in a sample, processes and devices for the improved blocking of non-target molecules and handling of precipitates would be advantageous.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by providing a fluidic device for detecting a target molecule in a fluid sample comprising a blocking chamber in fluidic communication with a detection chamber forming a blocking-detection chamber pair, the blocking chamber adapted to receive at least one reagent for binding a non-target molecule in the sample, the blocking chamber adapted to maintain at least a portion of the bound non-target molecule within the blocking chamber, the detection chamber adapted to receive at least one reagent for binding a target-molecule such that the target-molecule may be detected by binding to a detector. The device further comprises a combination detection chamber for receiving a second portion of the fluid sample, the combination detection chamber adapted to receive at least one reagent for binding both target and non-target molecules such that the combination of target and non-target molecules may be detected by binding to a detector

A method of detecting a target molecule in a fluid sample is also provided.

A method for detecting a total amount of target molecule in a sample is provided.

Use of the device and claimed methods for detecting a biomarker in a biological sample is also provided.

Further advantageous embodiments are disclosed in the appended and dependent patent claims.

### BRIEF DESCRIPTION OF THE DRAWINGS

These and other aspects, features and advantages of which the invention is capable will be apparent and elucidated from the following description of embodiments of the present invention, reference being made to the accompanying drawings, in which

FIG. 1 is a schematic top-view of a microfluidic device according to an aspect.

FIG. 2 is a schematic top-view of a microfluidic device according to an aspect, showing a detail view of a chamber connected to a siphon. The detail view is of the region Detail A encircled by the dotted line in FIG. 1.

FIG. 3 is a schematic top-view of a microfluidic device according to an aspect showing a detail of a portion comprising a plurality of blocking-detection chamber pairs. The detail view is of the region Detail B encircled by the dotted line in FIG. 1.

FIG. 4 is a schematic top-view of a microfluidic device according to an aspect showing the inner and outer radial edges with respect to the fluid handling features which are unnumbered.

### DETAILED DESCRIPTION

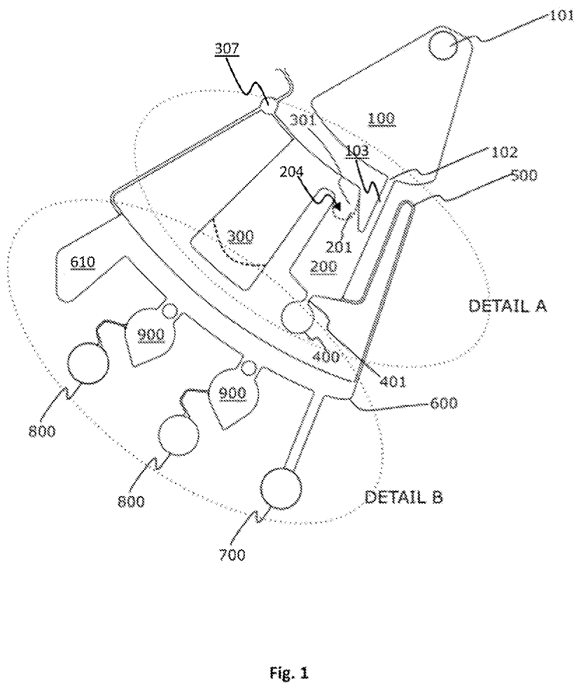

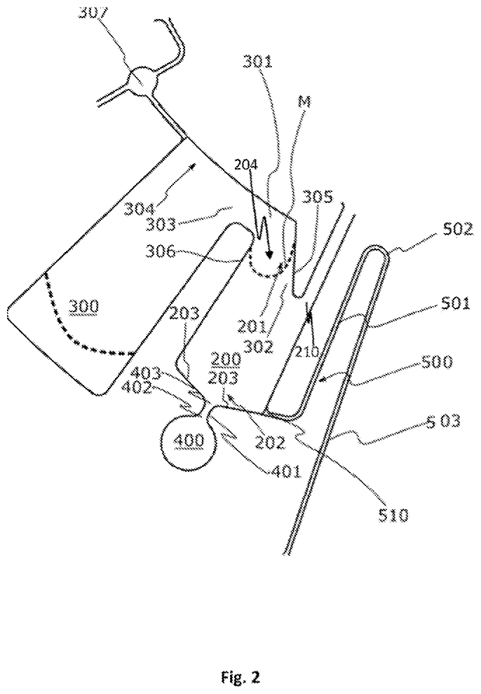

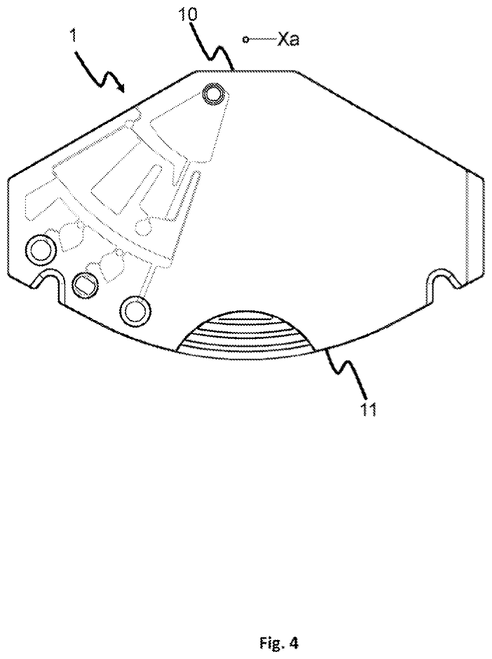

FIGS. 1-2 & 4 show a centrifugal microfluidic device 1 having an inner 10 and outer radial edges 11 (inner and outer edges shown in FIG. 4). To actively flow fluid through the channels and chambers of the microfluidic device 1 the device is generally rotated. The microfluidic device 1 is rotated around a central rotational axis Xa as shown in FIG. 4. The central rotational axis Xa is perpendicular to the plane of fluid flow in the microfluidic device. As the device 1 is rotated when in use, the device 1 may be considered to comprise a radial inner edge 10 and a radial outer edge 11. Portions or features of the device closer the radial inner edge 10 are considered radially inner portions or features. Portions or features which are closer to the radial outer edge 11 are considered radial outer portions or features. A radial outer feature is proximal the outer radial edge 11, distal the inner radial edge 10. A radial inner feature is proximal the radial inner edge 10, distal the radial outer edge 11. The radial inner edge 10 is proximal the central rotational axis Xa. The radial outer edge 11 is distal the central rotational axis. If an element or feature is described as being radially inward, or internal, of a second element, that refers to the first element's position being proximal the inner edge 10 with respect to the second element. If an element of feature is described as being radially external, or outward, of a second element, that refers to the first element's position being proximal the outer edge 11 with respect to the second element.

When the device 1 is not rotated the fluid in the device will settle, or will flow due to other forces than centrifugal force, such as capillary forces.

The microfluidic device 1 comprises a sample loading chamber 100 for receiving a sample. The sample loading chamber comprises a sample delivery port 101 via which a sample may be provided to the chamber 100. The sample delivery port 101 may be open to atmosphere. Generally the sample comprises a biological substance. The sample may comprise whole blood, serum, plasma, saliva, urine, or a combination thereof. The sample may be mixed with a buffer or reagent as is known in the art. The sample loading chamber 100 may have a volumetric capacity of about 10 to 100 μL, such as about 75 μL. The sample loading chamber 100 is fluidically connected to a metering chamber 200 for defining a volume of the sample which is deliverable to downstream features of the device 1. The metering chamber 200 has a volumetric capacity being less than the volumetric capacity of the sample loading chamber 100. The metering chamber 200 has a volumetric capacity less than the volume of sample generally loaded to the sample loading chamber 100. The metering chamber 200 is for metering a volume of fluid less than the total volume of the sample supplied to the device, the metered volume being for downstream use. Fluid is transferred to the metering chamber 200 from the sample loading chamber 100 via rotation of the microfluidic device at a set frequency such that the capillary stop formed at the outlet 102 of the first chamber is broken and fluid flows into the metering chamber 200. The fluid flows through a loading channel 103 which connects the sample loading chamber 100 to the metering chamber 200. The metering chamber 200 has a first opening 210 for receiving the sample via the loading channel 103. The loading channel 103 extends radially inward of the metering chamber 200 from the first opening 210 of the metering chamber 200. As would be understood, the specific frequency of rotation and duration is dependent on the dimensions of the channels and chambers. The microfluidic device as shown in FIG. 1 may be rotated at a frequency of about 1-100 Hz, such as about 40 Hz to actuate/displace fluid to the metering chamber 200.

The metering chamber 200 may be fluidically connected to an overflow chamber 300 which receives the volume of sample provided to the sample loading chamber 100 in excess of the volume of sample which may be received in the metering chamber 200. The overflow chamber 300 may be adjacent to and in connection to the metering chamber 200 via an overflow channel 301 provided at the radial inner portion 201 of the metering chamber 200. The overflow channel 301 is a relatively low fluidic resistance channel such that excess fluid may readily flow from the metering chamber 200 to the overflow chamber 300 when the fluid is filling the metering chamber 200 from the sample loading chamber 100. The inlet 302 to the overflow channel 301 from the metering chamber 200 is provided at the radial inner portion 201 of the metering chamber 200. The outlet 303 of the overflow channel 301 at the overflow chamber 300 is provided at the radial inner portion 304 of the overflow chamber 300. The inlet 302 to the overflow channel 301 may be substantially adjacent the inlet 102 to the metering chamber 200 from the sample loading chamber 100. A portion of the overflow channel 301 is inside radially with respect to the expected meniscus at the metering chamber 200 such that the volumes of fluid in the overflow chamber 300 and the metering chamber 200 are separate when the fluid has settled and is not flowing. That is, there are two separate volumes of fluid, a first volume in the metering chamber 200, and the excess in the overflow chamber 300. The two separate volumes of fluid are shown in FIGS. 1 and 2, the dashed lines in the metering chamber 200 and the overflow chamber 300 representing the meniscus of the two separate volumes of fluid.

The metering chamber 200 may be provided in connection with a vent 307 to atmosphere. The vent 307 to atmosphere may be in connection to the overflow chamber 300 and the metering chamber 200. The metering chamber 200 may be non-vented such that the device 1 is substantially sealed once sample has been provided to the sample delivery port 101. In such an arrangement a channel may be provided from the metering chamber 200, and/or the overflow chamber 300 to the sample loading chamber 100.

The metering chamber 200 may be provided in connection to a pellet chamber 400. The pellet chamber 400 may be provided in connection to the radial outermost portion of the metering chamber 200. If the sample comprises whole blood, such as in a diluted sample comprising whole blood, blood fractionation may occur and red blood cells (RBCs) may begin to separate from plasma or the diluted sample medium in the metering chamber 200. The pellet chamber 400 may be connected to the metering chamber 200 via a fractionation channel 401. The metering chamber 200 may comprise a radial outward portion which forms the pellet chamber 400. That is, the pellet chamber 400 need not be a separate chamber to the metering chamber 200, but may be a region of the metering chamber for collecting, for example, RBCs. To transfer the RBC layer from the metering chamber 200 to the pellet chamber 400 the microfluidic device 1 is rotated at a frequency which causes the fluidic closure at the outlet 402 of the fractionation channel 401 at the pellet chamber 400 to open. The pellet chamber 400 may be provided in connection with a vent. The vent enables air within the pellet chamber 400 to exit as the chamber 400 fills. The radial outermost walls 203 defining the radial outermost edge 203 of the metering chamber 200 may be angled such that the walls narrow and funnel towards the inlet 403 to the fractionation channel 401 at the metering chamber 200.

A metered volume of the fluidic sample, comprising a reduced portion of any precipitates, may exit the metering chamber 200 via a siphon 500 provided at the radial outer portion 202 of the metering chamber. Siphons are known in centrifugal microfluidics and are a passive valve which allow fluid to flow when the microfluidic device is rotated below a threshold frequency. The siphon 500 comprises at least two portions, a first portion 501 from an inlet 510 at the metering chamber 200, to a crest 502, the first portion 501 is directed inwards, from a radial outer position on the device to a radial inner position. The first portion 501 is for fluid flow in the opposite direction to the centrifugal force generated when the device 1 is rotated. The siphon 500 comprises a second portion 503 from the crest 502 to a subsequent, downstream, feature on the microfluidic device 1. The subsequent feature is radially outside the inlet 510 to the siphon 500 at the metering chamber 200. The second portion 503 is directed outwards, from a radial inner position on the device, to a radial outer position. The siphon 500 may be connected, either directly or indirectly to a vent downstream of the crest 502. Fluid flows through the siphon 500 due to capillary pressure and the hydrostatic pressure difference i.e., the fluid wicks along the siphon 500. When fluid is in the first portion 501 of the siphon 500, or is in the second portion 503 of the siphon 500 but is not radially outside the meniscus in the metering chamber 200 then rotation of the device 1 at a high rpm to induce centrifugal forces inhibits the flow of fluid within the siphon. Once fluid has passed the crest of the siphon 500, and is radially outside the gas-liquid interface(s) in the metering chamber 200 and/or the channel (103) then the siphon is “primed” and fluid may flow from the metering chamber 200 downstream.

One or more of the channel surfaces of the siphon 500 may be treated such that they are hydrophilic. However, generally the surfaces are not treated to simplify manufacturing. A surfactant or wetting agent may be added to the sample to reduce the surface tension of the fluid and encourage wetting. For example, a polysorbate type nonionic surfactant such as that marketed as Tween® 20 may be added to the sample. The siphon 500 may be considered a siphon channel. The term siphon as used herein refers to the channel having at least the two portions 501, 503 as described above connected via a crest 502.

Once fluid has passed the crest 502 of the siphon 500, and is radially outside the fluid level in the metering chamber 200, the siphon 500 is considered “primed” and the microfluidic device 1 may be rotated to flow fluid from the metering chamber 200 to the subsequent feature as described above.

The inlet 510 of the siphon is radially inside relative to the inlet 403 of the fractionation channel 401. The inlet 510 to the siphon 500 at the metering chamber 200 is provided at a position radially between the inlet 403 of the pellet chamber 400, and radially outside the inlet 302 to the overflow channel 301. If the device doesn't comprise a separate pellet chamber but rather a portion of the metering chamber 200 acts as a region for collecting a pellet, then the inlet 510 of the siphon 500 is radially inward of the region for collecting a pellet. To ensure that fluid doesn't flow from the metering chamber 200 to the siphon 500 during the delivery of the fluidic sample to the metering chamber 200, or during the fractionation, that is, during rotation, the first portion of the siphon 501 is directed inwards. As the siphon acts as a valve, rotation of the device 1 alone, to generate a hydrostatic pressure on the fluid, cannot force fluid into the siphon 500 from the metering chamber 200, as if this was the case, then the metering chamber 200 would be incapable of metering a defined volume of fluid during the fluid transfer step from the sample loading chamber 100 to the metering chamber 200.

As stated above, ideally, the fluidic sample wicks at least in part due to capillary pressure along the siphon 500 when the microfluidic device 1 is not rotating, or rotating at a sufficiently low speed such that the centrifugal forces are less than the capillary wicking pressure, however, in some cases the fluid may not wick, for example, due to insufficient capillary pressure. The problem of unreliable or degrading siphon valving performance is recognized in the art (Siegrist et al, Serial siphon valving for centrifugal microfluidic platforms. Microfluidics and Nanofluidics, 2010. Vol. 9, issue. 1, pp. 55-63). For example, the hydrophilic treatments provided to the surface of the siphon may degrade over time leading to reduced performance. Reduced performance in this respect means for example, none or very slow wicking of fluid along the siphon.

One technique proposed for improving the rate at which wicking occurs is oscillating acceleration or deceleration of the microfluidic device, using the principle of Euler forces acting on the fluid at the inlet to the siphon, may in some instances cause the fluid to flow into and prime the siphon. However, the inventors have found that this is unreliable process which may generate air bubbles which causes drag and limits the effect of such an oscillating process.

The inventors have identified that an improved technique to actuate fluid from the metering chamber 200 to the siphon 500 is use at least one of two separate, additional pressures to encourage and increase the rate of wicking.

The first being Laplace pressure formed at the liquid-gas interface M at the innermost portion 201 of the metering chamber 200 to force fluid into the siphon 500 from the metering chamber 200. The Laplace pressure is formed at a gas-liquid interface generating portion 204. The inlet 301 to the overflow chamber 300 from the metering chamber 200 is formed by a channel having two side-walls 305, 306. One of the two side walls 305, 306 is angled with respect to the other side-wall 305, 306 such that the channel 301 narrows in the radial inwards direction. This angled side-wall 305, 306 leads to the meniscus M at the liquid-gas interface M at the innermost portion 201 of the metering chamber 200 to have a significantly reduced radius with respect to the meniscus which would form if the side-walls were parallel. The meniscus M can be seen as the dashed line in the metering chamber in FIG. 2. The Laplace pressure formed at the liquid-gas interface M is inversely proportional to the radius of meniscus, that is, a reduced radius of the meniscus results in a greater Laplace pressure. The radius of the meniscus is defined by the geometry of the overflow channel 301 and the contact angle of the fluid at the walls 305, 306 of the channel 301. By angling at least one of the side-walls 305 306 such that the walls 305, 306 of the overflow channel 301 form a narrow region at the inner portion 201 of the metering chamber 200, the radius of the meniscus M is significantly reduced with respect to having a channel with parallel side-walls.

The fluid is thereby passively encouraged to flow into, and through, the siphon 500 via altering the geometry of a feature neither in the siphon 500, nor at the siphon inlet 510.

As can be seen in FIG. 1, the liquid-gas interface M at the innermost portion 201 of the metering chamber 200 is separate to any interface formed in the loading channel 103. The angled side-wall 305, 306 separates the liquid-gas interface M at the innermost portion 201 of the metering chamber 200 from any interface formed at the loading channel 103. This enables the two separate pressures to encourage and increase the rate of wicking.

A second additional pressure is the hydrostatic pressure of gas-liquid interface in the channel 103. That is, the gas-liquid interface radially inward of the gas-liquid interface of fluid in the siphon 500.

The provision of the first or second, or most preferably both the first and the second additional pressures assist the capillary pressure in wicking the fluid through the siphon 500. This increase in wicking rate allows more rapid fluid handling operations.

The microfluidic device 1 is rotated at a low frequency such that the centrifugal force acting on the sample in the metering chamber 200 overcomes the tendency of the fluid to wick due to capillary forces completely along the wetted loading channel 103 into the sample loading chamber 100, and/or along the side-walls 305, 306 of the overflow channel 301.

Whilst the gas-liquid interface generating portion 204 has been described above with respect to metering chamber 200 and the overflow chamber 301, the gas-liquid interface generating portion 204 need not be specific to the metering chamber 200, nor the overflow channel 301 and chamber 300, of the microfluidic device 1. The metering chamber 200 could be a chamber provided with an inlet to a siphon 500. The gas-liquid interface generating portion 204 need not be a portion of the overflow channel 301. The gas-liquid interface generating portion 204 may be a portion of a chamber in connection to a siphon 500, wherein the portion is formed by side-walls, at least one of which is angled with respect to the other, such that the portion narrows in the opposite direction to the direction in which centrifugal forces are applied to a fluid.

The low frequency is less than the frequency required to break or re-break the fluidic seal at the inlet 403 to the fractionation channel 401 at the metering chamber 200 but sufficient to actuate the fluid into the siphon 500. The rotational frequency may for example be about 1 Hz.

The angled side-wall 305, 306 of the overflow channel 301 at the gas-liquid interface generating portion 204, forming the inner meniscus M, in combination with the hydrostatic pressure of the gas-liquid interface in the channel 103 with respect to the gas-liquid interface in the siphon 500, and the low frequency rotation, thereby causes a mass transfer to the siphon 500. The rate at which fluid may flow along the siphon 500 compared to allowing the fluidic sample to wick without the portion 204 and/or the channel 103, in combination with rotation below a threshold frequency is increased or without the gas-liquid interface generating portion 204.

In the above arrangement there are three separate interfaces between air and the liquid sample: a first interface at the loading channel 103, a second interface at the at the gas-liquid interface generating portion 204, the interface formed referred to as M, and a third interface in the siphon (not-shown).

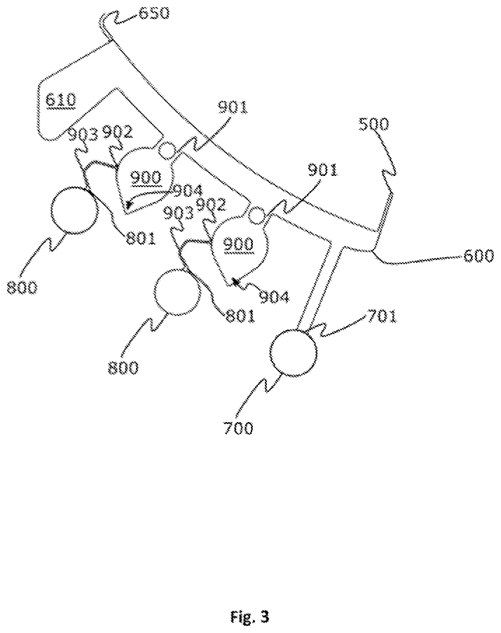

FIGS. 1, 3-4 show the microfluidic device 1 comprising at least one, such as a plurality of detection chambers 700, 800, each chamber for detecting an analyte. The analyte detected in each chamber 700, 800 may be a different analyte, or it may be the same in one or more of the detection chambers 700, 800. The chambers 700, 800 may be, but need not necessarily be, dead-end chambers which are not vented and from which fluid may enter from a single inlet 701, 801 provided respectively to each dead-end chamber 700, 800. Each of the detection chambers 700, 800 may be provided with a reagent which mixes with the fluidic sample on delivery of the fluidic sample to the respective chamber. If the chambers 700, 800 are non-vented then the duration which the sample is receivable in the chambers 700, 800 and the blocking chamber 900 is controllable via the speed of rotation of the microfluidic device 1, and therefore sedimentation or palletisation time are better controllable.

Each detection chamber 700, 800 may be used to detect an analyte. Magnetic nanoparticles may be present in each of the detection chambers. A biosensor for the detection of analytes using functionalized magnetic nanoparticles is described in EP 3 014 245 B 1. In EP 3 014 245 B 1 the magnetic nanoparticles which have been functionalized with bioactive ligands is described. Each of the plurality of detection chambers 700, 800 may comprise functionalized nanoparticles as described therein.

When detecting analytes, it may be desirable to block and/or precipitate specific components from the sample such that they do not interfere with the detection of target analyte(s) and measurement processes. For example, it may be desirable to block a specific antibody from the sample such that it will not interfere with a detection process for another antibody. Generally, the sample may comprise, for each detection process, a target molecule, i.e., an analyte and at least one non-target molecule. The non-target molecule may otherwise impact the detection and measurement if it is present during the detection process. For example, the non-target molecule may bind non-specifically to a detector. A blocking process is a process whereby non-target molecules are selectively blocked and/or precipitated such that the target molecule remains in solution. A blocking medium may comprise a specific blocking molecule selected to precipitate a non-target molecule.

A problem with the typical blocking process based on immunoprecipitation is that it requires optimisation of the point of equivalence for optimal antigen and antibody interaction. That is the concentrations of the blocking molecule in the blocking medium must be matched to the concentration of non-target molecules in the sample. However, the concentration of non-target molecules in the sample is generally not known in advance. An inadequate amount of blocking molecule leads to insufficient blocking of the non-target molecules. An excess of blocking molecule may lead to the formation of soluble complexes of the non-target molecule, saturated with the blocking molecule, which does not precipitate and nor block optimally. The non-blocked non-target molecule interferes with measurement processes. This is especially a problem in immunoassays where the presence and quantity of specific antibodies are to be determined, and where optimal antibody concentration must generally be determined through experimentation—which is not possible if a rapid result is desired with an unknown sample concentration.

The inventors have identified that the above problem may be overcome or alleviated by using a blocking chamber 900 and a detection chamber 800 provided to the microfluidic device 1.

The microfluidic device 1 is provided with at least one blocking chamber 900 in fluidic connection with one detection chamber 800 forming a blocking-detection chamber pair 800, 900. The blocking chamber 900 is for blocking and precipitating a specific component of the sample, and maintaining it within the chamber such that the precipitated component does not enter the detection chamber 800. Each blocking chamber 900 may be provided in connection to a detection chamber 900, forming a blocking-detection chamber pair 800, 900. The microfluidic device may comprise a plurality of blocking-detection chamber pairs 800, 900. For each of the blocking-detection pairs 800, 900, the sample may have to flow through the blocking chamber 900 to enter the detection chamber 800. The blocking chambers 900 may be fluidically connected to a manifold 600 which delivers fluid to the blocking chambers 900 and/or detection chambers 800, 700. The blocking chamber 900 is fluidically connected to the manifold 600, and to the detection chamber 800. At least one of the detection chambers 700 may be provided without being connected to a blocking chamber. That is, at least one of the detection chambers 700 may be directly fluidically connected to the manifold 600 without an intermediate blocking chamber. This directly connected detection chamber 700 may be suitable for detecting an analyte for which the blocking of other analytes is not necessary for a detection process.

The blocking chamber 900 comprises an inlet 901 at its radial inner portion for receiving the sample. The blocking chamber 900 further comprises an outlet 902 to the transfer channel 903 which connects the blocking chamber 900 to the detection chamber 800. The outlet 902 to the transfer channel 903 is provided at a region between the radial outermost portion of the blocking chamber 900, and the radial innermost portion, such that the blocking chamber comprises a radial outer region 904, radially outside the outlet 902, for receiving and maintaining precipitate separate from the outlet 902 to the transfer channel 903. The blocking chamber 900 may comprise a tapered radial outer portion 904 forming a funnel-like shape for receiving precipitate. The outlet 902 to the transfer channel 903 is radially inward of the portion 904 for receiving and containing precipitate. The portion 904 may be referred as the precipitate capture region 904.

A sample entering the blocking chamber 900 mixes with a reagent present in the blocking chamber 900. The reagent may be a dried spot of reagent present, for example, on the bottom surface of the blocking chamber 900. The reagent may be mixed with the fluidic sample within the blocking chamber 900. The reagent may comprise a buffer and various other components. The reagent may comprise at least one component selected to bind to a non-target molecule and/or cause a non-target molecule within the sample to precipitate. For example, the reagent may comprise an antibody which forms an insoluble antibody-antibody complex with an antibody present in the sample. Precipitate may form in the blocking chamber 900.

Mixing the sample and the reagent in the blocking chamber 900 causes a proportion, such as a majority, of the non-target molecule within the sample to be blocked such that it cannot bind or otherwise interfere with the detection process. A proportion, such as a majority of the non-target molecule may be precipitated. The supernatant may be partially or substantially free of the non-target molecule.

Fluid enters the blocking chamber 900 when the device is rotated at a frequency such that the fluidic stop at the inlet 901 is broken. Fluid then flows into and at least partially fills the blocking chamber 900. Without being bound by theory the inventors have recognised that due to the centrifugal forces acting on the fluid in the chamber 900 during the filling there may occur a concentration gradient of blocking agent, from the dissolved reagent spot. The concentration gradient is such that the radial outer portion of the blocking chamber 900 has the highest concentration of blocking agent, the radial inner portion of the blocking chamber 900 has the lowest concentration. This gradient of concentration may lead to specific bands of ideal concentrations for precipitation, zones of equivalence, forming within the blocking chamber 900.

After the precipitate has formed, the blocking chamber 900 comprises a precipitate comprising a non-target molecule, and a supernatant comprising a target molecule. As would be understood, the supernatant may comprise other different non-target molecules, a small amount of non-target which was not blocked/remains unprecipitated in the first precipitation step and/or buffers etc.

To ensure that the precipitate does not enter the detection chamber 800 the microfluidic device 1 is rotated at a frequency sufficient to displace via centrifugal force the precipitate to the precipitate receiving and containing portion 904 of the blocking chamber. The rotational frequency is less than the frequency required to overcome the burst pressure at the fluidic closure generated by the meniscus at the inlet 801 to the detection chamber 800 from the transfer channel 903. The fluidic closure may, in some instances, form at the outlet 902 of the blocking chamber 900 to the transfer channel 903. This rotation causes the precipitate in the blocking chamber 900 to gather in the radial outermost portion 904, and be maintained separate from the outlet 902 to the transfer channel 903 and therein the detection chamber 800.

To force the supernatant into the transfer channel 903 and detection chamber 800 the microfluidic device 1 may be rotated at a frequency sufficient to overcome the burst pressure at the fluidic closure generated by the inlet 801 to the detection chamber 800.

The detection chamber 800 may comprise, as stated above, functionalized magnetic nanoparticles. In addition to the blocking chamber 900, the detection chamber 800 may comprise reagents comprising at least one component selected to block and/or precipitate a component in the supernatant. The blocking process then becomes a two-step blocking process, the first step being in the blocking chamber 900, and the second step in the detection chamber 900.

The reagent in the second step of the two-step precipitation process may comprise at least one component selected to block and/or precipitate at least one of: the same non-target molecule that was precipitated in the first step, a different non-target molecule to that which was precipitated in the first step, complexes of partially blocked but unprecipitated molecules from the first step.

The two-step process will now be exemplified with respect to human immunoglobulin G (IgG) blocking to measure immunoglobulin A (IgA) in a human blood sample in the microfluidic device 1. The process is substantially the same for detection of, for example, immunoglobulin M (IgM).

A sample comprising at least IgG & IgA flows into the manifold 600. A plurality of detection chambers 700, 800 are connected to the manifold, either via blocking chambers 900 or directly. A first detection chamber 700 is directly connected to the manifold 600. The sample flows into the first detection chamber 700. The first detection chamber 700 is provided with a plurality of functionalized magnetic nanoparticles adapted for detecting IgG as part of a detection system such as that described in EP 3 014 245 B1. The first detection chamber 700 may also be provided with a reagent, such as a dried reagent which mixes with the fluidic sample within the detection chamber 700. The amount of anti-target IgG in the sample in the first detection chamber 700 is therein determined. The detection process may not be fully specific to IgG, such that the amount of IgG determined in this step may comprise the amount of IgG, IgM and/or IgA etc. antibodies present in the sample. The first chamber 700 may detect a total amount of human antibodies, or a portion thereof, present in the sample.

As the first chamber 700 detects the total amount of antibodies and may not be specific to IgG, IgM and/or IgA the first chamber may be referred to as the combination detection chamber 700. As described above, the combination detection chamber 700 need not only be for detecting human antibodies, other analytes not being human antibodies may be detected in combination in the combination detection chamber 700.

As is described above, a first portion of the sample may be received in the combination detection chamber 700. A second portion of the sample may be received in the blocking-detection chamber pair 900, 800. The first portion may be separate from the second portion, that is, once a portion is received in the combination detection chamber 700 it is not subsequently receivable in the blocking-detection chamber pair 800, 900. This allows, as is described herein, the measurement of a total amount of analytes being both target and non-target in the combination detection chamber 700, and an amount of target analyte in the detection chamber 800, after blocking in the blocking chamber 900.

There may be other methods for determining the amount of IgG in the sample besides functionalized nanoparticles as described in EP 3 014 245 B 1.

The sample flows from the manifold 600 to a blocking-detection 900, 800 chamber pair. As described above, the sample flows first to the blocking chamber 900 of the blocking-detection pair 900, 800. The blocking chamber 900 is provided with a reagent such as a buffer comprising anti-human IgG antibodies. The sample mixes with the reagent and a portion, such a majority of the IgG present in the sample is blocked such that it cannot bind to magnetic nanoparticles in the detection chamber. A precipitate comprising blocked IgG and anti-IgG complexes bound together forms in the blocking chamber 900, the precipitate may form throughout the blocking chamber 900 and need not be located in a specific region. However, as described above, as due to the radially increasing concentration of anti-human IgG antibodies, i.e., blocking molecule, the precipitate may form in a radial band within the blocking chamber. The microfluidic device 1 is rotated at a first frequency such that centrifugal force acts on the precipitate within the blocking chamber 900. The frequency generates a pressure at the outlet 801 to the detection chamber 800 from the transfer channel 903 which is less than the pressure required to open the fluidic closure formed by the meniscus, the burst pressure. The precipitate flows into the precipitate capture region 904 being rotationally outward with respect to the inlet 902 to the transfer channel 903.

The microfluidic device 1 can thereafter be rotated at a second frequency sufficient to open the fluidic closure formed by the meniscus at the outlet to the detection chamber 800 from the transfer channel 903, the second frequency greater than the first frequency for displacing the precipitate to the precipitate capture region 904. The supernatant flows from the blocking chamber 900 to the detection chamber 800 of the blocking-detection 900, 800 chamber pair.

The detection chamber 800 comprises functionalized magnetic nanoparticles and a reagent. The reagent may comprise a buffer and, in some cases, additional anti-human IgG antibodies for two-step blocking. On mixing of the supernatant with the reagent, any remaining IgG in the supernatant may be bound by the anti-human IgG antibodies. As there will be substantially less IgG in the supernatant than in the sample less anti-human IgG is required in the detection chamber 800 than in the blocking chamber 900.

The detection chamber 800 is provided with a plurality of functionalized magnetic nanoparticles adapted for detecting IgA as part of a detection system such as that described in EP 3 014 245 B 1. As the IgG has been blocked by anti-human IgG in the blocking chamber 900 prior to being provided to the detection chamber 800 there is substantially less precipitate than may otherwise be present in the detection chamber 800, the amount of IgA may be accurately determined by a detection system.

As stated above, neither the blocking process described above, nor the blocking-detection chamber pair 900, 800 require a detection system based on magnetic nanoparticles. Other detection systems for detecting analytes may be used in the detection chamber. However, the removal of the blocking and precipitate prior to provision of the sample to the detection chamber is especially suitable in testing and detection methods where both non-blocked non-target molecules, and precipitate itself, could disturb and reduce measurement performance.

The detection process may include a step for comparing the amount of IgG measured in the sample to the amount of IgA. As described above, IgA may not have been blocked during the IgG detection step. Therefore, the total amount of IgG measured may include a portion of non-specifically bound IgA. However, the amount of IgA in the sample is known from the IgA detection step. Therefore, with the above process, the total amount of IgG may be calculation according to the following formula:

Actual IgG=[IgG measured in IgG step]−k*[IgA measured in IgA step]

Where k is a constant determined and verified through experimentation.

The process described herein is especially suitable for detecting human antibodies, and in particular IgA and IgM whereby removal or blocking of IgG is necessary. However, other analytes may be blocked, and detected by the two-step process.

The formula may therefore be generalised to:

Total Molecule of Interest=[total target molecule & non-target molecule measured in combination chamber]−k*[target molecule measured in detection chamber]

Or, if a plurality of blocking-detection chambers are present in the device, where each pair is adapted to measure a specific target molecule, and therein block a separate non-target molecule, and where a total amount is measured in the combination chamber:

Total Molecule of Interest=[total target molecule & non-target molecules₁₋ₓ measured in combination chamber]−k₁*[target molecule₁ measured in a detection chamber₁] . . . −kₓ*[target moleculeₓmeasured in detection chamberₓ]

For clarification, the term “target” as used herein does not refer to the molecule of interest but rather the target molecule detected in the detection chamber 800 after the blocking chamber, and therein the blocking step in a method. As is described above in the example with human IgG and IgA, IgG is the molecule of interest, and IgA is the target molecule.

The amount or concentration of the total molecule of interest e.g., IgG in the fluid sample, may be used diagnose an infection an in a blood sample from a mammal.

The above process has been described in conjunction with a blocking chamber comprising a region 904 for collecting precipitate radially external to the inlet 902 to the transfer channel 903. However, it is possible that other aspects of the blocking step in the process may use for example, physical entrapment of the blocked antibodies in the blocking chamber. For example, the blocking chamber 900 may comprise beads functionalized with molecules which bind and attach the non-target molecules. In such an aspect the beads have a diameter greater than the width of the inlet 902 to the transfer channel 903 such that the beads do not flow into the transfer channel 903. The blocking chamber 900 may comprise a functionalized filter or membrane which traps non-target molecules. At least one surface of the blocking chamber 900 may comprise molecules which bind to and physically hold the non-target molecules. The above aspects trap, ideally a substantial proportion of, such as a majority of the non-target molecules in the blocking chamber 900.

The manifold 600 may be provided in connection to a vent 650. The vent 650 may be the same, or fluidically connected to the vent 307 which is connected to the metering chamber 200. Instead of a vent, the manifold 600 may be provided with a channel connecting the manifold 600 to the sample loading chamber 100. In such a manner a closed system is provided with no venting.

The manifold 600 may be provided with a second overflow chamber 610. The second overflow chamber 610 receives the volume of sample in excess of that which can be comprised in the detection chamber(s) 700, 800 and blocking chamber(s) 900.

Such that the detection chamber 700, 800 can be used with a light-based detection system, at least the base of the detection chambers 700, 800 is substantially transparent to light in the visible and ultraviolet wavelengths. Light is directed to the base and/or the top of the detection chamber 700, 800 and is transmitted through the base and/or top of the detection chamber 700, 800 to, for example, magnetic nanoparticles therein. An aperture may be provided above, or below the detection chambers 700, 800 coaxial with the centre of the chamber 700, 800. The aperture may have at least one angled wall with respect to the Z plane (into the page in FIGS. 1-3) such that incident light not directed at the radial centre of the detection chamber 700, 800 is reflected away and is not transmitted to the magnetic nanoparticles. This may improve the quality of the detected signal as only sample in the centre of the detection chamber 700, 800 receives light.

The microfluidic device 1 comprises a substrate and a cover. As would be understood the fluidic structures such as channels, chambers, vents etc. may be provided in either the substrate, the cover, or both. The fluidics structures are substantially within the same plane, however, certain fluidic structures may be in, for example, the cover, and certain fluidic structures may be, for example in the substrate, leading to them being in different, but adjacent planes.

The microfluidic device 1 comprises structures in the micrometre range such that volumes of liquids in the nanolitre to microlitre range may be processed on the device 1. The term fluid used herein is used in the sense of a microfluidic device and therefore refers generally to a liquid fluid. If a gas fluid is meant, such as air, then it is referred to as a gas.

The microfluidic device 1 may be formed from any suitable material, such as a glass or plastic. Suitable plastics may for example be PMMA, PC, PVC, or PDMS etc.

Although, the present invention has been described above with reference to specific embodiments, it is not intended to be limited to the specific form set forth herein. Rather, the invention is limited only by the accompanying claims.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

Experimental Section

Semi-quantitative detection of human IgA+IgM and IgG antibodies directed against SARS-CoV-2 in human whole blood and plasma.

General Process

The microfluidic device 1 as described herein, designated ViroTrack Sero COVID-19 IgA+IgM/IgG Ab was used throughout the following experimental section.

10 μL of sample was collected with a sample collection pipette and transferred to a dilution buffer in a vial comprising 150 μL of dilution buffer comprising 0.09% sodium azide.

Subsequently 50 μL of the diluted sample was dispensed into the microfluidic device 1 via the sample delivery port 101. The microfluidic device 1 was then inserted into the BluBox (BluSense Diagnostics ApS) for centrifugal actuation and analysis.

The microfluidic device 1 metered the diluted sample via the metering chamber 200 and red blood cells present in the sample were fractionated to the pellet chamber 400.

For IgG detection, the sample was transferred, via rotation of the microfluidic device 1, to the combination detection chamber 700 where at least IgG was measured. The combination detection chamber 700 comprised magnetic nanoparticles coated with SARS-CoV-2 antigens.

For IgA+IgM detection, the sample was first transferred, via rotation of the microfluidic device 1, to the blocking chamber 900 where a dried blocking buffer was dissolved, blocking at least a portion of the IgG present in the sample. The sample was then transferred to the detection chamber 800. The detection chamber 800 comprised magnetic nanoparticles coated with SARS-CoV-2 antigens.

In both the combination detection chamber 700 and the detection chamber 800, the SARS-CoV-2 specific antibodies bound to the SARS-CoV-2 antigens immobilized on the surfaces of the magnetic nanoparticles. The binding caused the agglutination of the magnetic nanoparticles, which was subsequently measured by the BluBox using the optomagnetic based detection system described in EP 3 014 245 B1.

The IgG amount was calculated by subtracting the measured IgM amount.

A semi-quantitative unit IMA was determined for each sample. The IMA unit reflects the reactivity of IgA+IgM or IgG anti-SARS-CoV-2 antibodies in the sample in a concentration-dependent manner.

Cross Reactivity of the IgA+IgM/IgG Detection Process

Cross reactivity of the detection process described above was evaluated using serum or plasma samples tested positives for other pathogens. A total of 70 patient samples were investigated. The results are shown in the table below.

| Infectious agents | Number of | Reactive to ViroTrack Sero COVID-19 IgA + IgM/IgG Ab | |
|---|---|---|---|
| or control serum | samples | IgG | IgA + IgM |
| Adenovirus/Influenza A/Influenza | 11 | 0% | 0% |
| B/Coxsackievirus/Echovirus | | | |
| Rheumatoid factor (RF) | 5 | 0% | 0% |
| Human Anti Mouse Antibody (HAMA) | 3 | | |
| Zika | 13 | 0% | 0% |
| Dengue | 10 | 0% | 0% |
| Malaria | 3 | 0% | 0% |
| Hepatitis A | 5 | 0% | 0% |
| Hepatitis B | 5 | 0% | 0% |
| Hepatitis C | 5 | 0% | 0% |
| Human immunodeficiency virus (HIV) | 5 | 0% | 0% |
| Cytomegalovirus | 5 | 0% | 0% |

Interference

Potential endogenous interference of the ViroTrack Sero COVID-19 IgA+IgM/IgG Ab was evaluated by spiking potential interferents at different concentrations into negative sample and positive sample for SARS-CoV-2 IgG antibodies. No significant interferences were observed at the stated substances and concentrations: Hemoglobin 10 g/L; Unconjugated Bilirubin up to 40 mg/dL; Conjugated Bilirubin up to 40 mg/dL; Triglycerides up to 1500 mg/dL.

Sensitivity-Specificity

The ViroTrack Sero COVID-19 IgA+IgM/IgG Ab was referenced to PCR and compared to a commercial ELISA kit for 122 samples (35 POS, 87 NEG). The results are show in the table below.

| | ELISA | | | ViroTrack Sero COVID-19 IgA + IgM/IgG Ab | | |
|---|---|---|---|---|---|---|
| | IgA | IgG | IgA + IgG | IgA + IgM | IgG | IgA + IgM/IgG |
| Sensitivity | 29/35 | 29/35 | 29/35 | 28/35 | 27/35 | 29/35 |
| | (82.9%) | (82.9%) | (82.9%) | (80%) | (77.1%) | (82.9%) |
| Specificity | 85/87 | 86/87 | 85/87 | 86/87 | 87/87 | 86/87 |
| | (97.7%) | (98.9%) | (97.7%) | (98.9%) | (100%) | (98.9%) |
| Agreement | 114/122 | 115/122 | 114/122 | 114/122 | 114/122 | 115/122 |
| | (93.4%) | (94.3%) | (93.4%) | (93.4%) | (93.4%) | (94.3%) |

Positive Agreement According to Days Post Onset of Symptoms

Positive agreement was evaluated using 33 plasma samples collected at different days from symptoms onset from subjects confirmed positive for SARS-CoV-2 by PCR.

| Days from | | | ViroTrack Sero COVID-19 IgA + IgM/IgG Ab results | | |
|---|---|---|---|---|---|
| symptom onset to | Number of | PCR | IgA + IgM | IgG | IgA + IgM/IgG |
| blood collection | samples | Result | (+) | (+) | (+) |
| 1-8 days | 16 | POS | 13/16 | 12/16 | 13/16 |
| | | | (81.2%) | (75%) | (81.2%) |
| >8 days | 17 | POS | 15/17 | 15/17 | 16/17 |
| | | | (88.2%) | (88.2%) | (94.1%) |
| Total | 33 | N/A | 28/33 | 27/33 | 29/33 |
| | | | (84.8%) | (81.8%) | (87.9%) |

IMA Quantification Across Serially Collected Samples

Plasma sample from two (2) symptomatic patients confirmed positive by SARS-CoV-2 PCR were collected serially every 1-5 days after the first blood draws. Samples were tested with ViroTrack Sero COVID-19 IgA+IgM/IgG Ab. IgA+IgM and IgG quantification results in IMA units are presented in the table below.

| Serial blood | Patient 1 | | Patient 2 | |
|---|---|---|---|---|
| collection | IgA + IgM | Ig G | IgA + IgM | Ig G |
| draws | IMA unit | IMA unit | IMA unit | IMA unit |
| Draw 1 | 1.6 | 2.3 | 2.5 | 1.3 |
| Draw 2 | 6.3 | 6.0 | 47.1 | 22.6 |
| Draw 3 | 29.5 | 18.0 | 36.5 | 21.7 |
| Draw 4 | 26.4 | 19.4 | 35.5 | 23.4 |

Dilution of Positive Samples

Plasma sample from three (3) patients confirmed positive by SARS-CoV-2 PCR were pooled, serially diluted, and tested with ViroTrack Sero COVID-19 IgA+IgM/IgG Ab. Results in IMA units are presented in the table below.

| Dilution | 1:1 | 1:3 | 1:9 | 1:27 | 1:81 | 1:243 |
|---|---|---|---|---|---|---|
| IgA + IgM IMA unit | 68.0 | 27.5 | 9.1 | 3.9 | 2.7 | 2.0 |
| IgG IMA unit | 19.5 | 10.9 | 5.9 | 4.4 | 2.4 | 2.0 |

The experiments show that the microfluidic device 1 is a simple-to-use point of care test device with sensitivity, specificity similar to commercial ELISA.

1.-17. (canceled)

18. A fluidic device for detecting a target molecule in a fluid sample comprising:
a blocking chamber in fluidic communication with a detection chamber forming a blocking-detection chamber pair for receiving a first portion of the sample, the blocking chamber adapted to receive at least one reagent for binding a non-target molecule in the sample, the blocking chamber adapted to maintain at least a portion of the bound non-target molecule within the blocking chamber, the detection chamber adapted to receive at least one reagent for binding a target-molecule such that the target-molecule may be detected by binding to a detector; and,
a combination detection chamber for receiving a second portion of the fluid sample, the combination detection chamber adapted to receive at least one reagent for binding both target and non-target molecules such that the combination of target and non-target molecules may be detected by binding to a detector.

19. The fluidic device according to claim 18, wherein the detection chamber is further adapted to receive at least one reagent for binding a non-target molecule in the sample.

20. The fluidic device according to claim 18, wherein the blocking chamber and the detection chamber comprise respective blocking agents.

21. The fluidic device according to claim 18, wherein the blocking chamber is fluidically connected to the detection chamber via a channel, and wherein the blocking chamber comprises a portion for receiving and maintaining a precipitate.

22. The fluidic device according to claim 18, wherein the combination detection chamber is arranged such that the portion of the fluid sample receivable in the combination detection chamber is not receivable in a blocking chamber.

23. The fluidic device according to claim 18, wherein the device is a centrifugal microfluidic device having a radial inner portion and a radial outer portion, and wherein the portion for receiving and maintaining the precipitate is arranged radially outward of an opening to the channel from the blocking chamber.

24. The centrifugal microfluidic device according to claim 23, wherein the portion for receiving and maintaining the precipitate is defined by a pair of side-walls which taper towards each other in the direction of the radial outer portion of the device.

25. The fluidic device according to claim 18, wherein the detection chamber and/or the combination detection chamber are adapted to receive functionalised magnetic nanoparticles adapted to bind to the target molecule.

26. The fluidic device according to claim 18, wherein the blocking-detection chamber pair are in fluidic communication with a manifold.

27. The fluidic device according to claim 26, wherein the device comprises a plurality of blocking-detection chamber pairs, each of the blocking chambers of the pairs respectively connected in parallel via the fluidic manifold.

28. The fluidic device according to claim 26, wherein the combination detection chamber and the blocking-detection chamber pair are in fluidic communication with the manifold.

29. The fluidic device according to claim 27, wherein at least two of the detection chambers are adapted to detect different target molecules.

30. A method of detecting a target molecule in a fluid sample, the fluid sample comprising a target molecule and at least one non-target molecule, the method comprising:
mixing a portion of the fluid sample with a reagent comprising a component adapted to bind and/or precipitate a portion of at least one of the non-target molecules,
capturing and maintaining the bound non-target molecules in a first blocking chamber of a fluidic device,
transferring the portion of fluid comprising the target-molecule to a detection chamber of a fluidic device,
detecting the target molecule in the detection chamber.

31. The method according to claim 30, wherein the method further comprises:
receiving a second portion of the fluid sample in a combination detection chamber of a fluidic device,
detecting both target molecules and non-target molecules in the combination detection chamber.

32. The method according to claim 31, wherein the method further comprises:
determining the total amount of a molecule in the fluid sample by comparing the detected amount of target and non-target molecules in the combination detection chamber to the detected amount of target molecules in the detection chamber.

33. A method of detection a target biomarker in a biological sample comprising providing the biological sample to a fluidic device for detecting a target molecule in a fluid sample according to claim 18.

34. The method according to claim 32, wherein the method is for the detection of SARS-CoV-2 antibodies in a sample, and wherein the combination detection chamber is provided with SARS-CoV-2 antigens such that total SARS-CoV-2 antibodies are detectable, and wherein the detection chamber is provided with SARS-CoV-2 antigens, and wherein the blocking chamber is provided with an IgG blocking agent.

35. The method according to claim 30, wherein the method is adapted for detection of a target biomarker in a biological sample.

36. The method according to claim 32, wherein the method is adapted for detection of a target biomarker in a biological sample.
